(19) United States
(12) Patent Application Publication
(10) Pub. No.: US 2005/0232904 A1
(43) Pub. Date: Oct. 20, 2005

(54) Methods of utilizing cultured hematopoietic progenitor cells for inducing immunological tolerance

(75) Inventors: Yair Reisner, Tel-Aviv (IL); Hilit Gur, Ness Ziona (IL)

Correspondence Address: Martin Moynihan;c/o Anthony Castorina, Suite 207, 2001 Jefferson Davis Highway, Arlington, VA, 22202, US

(73) Assignee: Yeda Research and Development Co. Ltd., Rehovot (IL)

(21) Appl. No.: 11/090,189

(22) Filed: Mar. 28, 2005

Related U.S. Application Data
(63) Continuation of application No. 09/881,721, filed on Jun. 18, 2001.

(51) Int. Cl. A61K 48/00; C12N 5/08

(52) U.S. Cl. 424/93.21; 435/372

(57) ABSTRACT

A method of inducing tolerance to a transplant transplanted from a donor to a recipient is disclosed. The method comprises (a) culturing a hematopoietic progenitor cell (HPC) population under growth conditions suitable for inducing or enhancing veto activity in at least a portion of the HPC population, thereby generating a tolerance-inducing cell population; and (b) administering a dose of the tolerance-inducing cell population prior to, concomitantly with or following transplantation of the transplant, thereby inducing tolerance to the transplant in the recipient.

This is a continuation of U.S. patent application Ser. No. 09/881,721, filed Jun. 18, 2001.

### FIELD AND BACKGROUND OF THE INVENTION

The present invention relates to methods of inducing immunological tolerance. More particularly, the present invention relates to the use of cultured hematopoietic progenitor cells (HPCs) for inducing durable tolerance to transplants transplanted across major histocompatibility barriers without risk of inducing graft versus host disease (GVHD). The present invention also relates to the use of such cells in alleviating or treating autoimmune diseases. The present invention also relates to methods of predicting the transplant tolerance-inducing activity possessed by preparations of cultured HPCs and methods of isolating cells possessing enhanced tolerance-inducing activity relative to non-cultured HPCs from such cell preparations.

Transplantation of allogeneic and xenogeneic organs, tissues and cells is commonly practiced in humans in order to alleviate numerous disorders and diseases. For example, bone marrow (BM) transplantation (BMT) is increasingly used to treat a series of severe diseases in humans, such as leukemia. However, BMT is limited by the availability of major histocompatibility complex (MHC) histocompatible donors, since transplantation between non-MHC histocompatible donors and recipients leads to graft rejection or GVHD. Such graft rejection may arise from the marked level of host hematopoietic and immune cells surviving in recipient patients conditioned sublethally. In view of such limitations, several approaches for enhancing graft acceptance have been suggested or are in application.

One approach for enhancing graft acceptance is based on the use of donor-derived cells possessing veto activity, which are termed “veto cells”, and are capable of inducing recipient tolerance to transplant from the veto cell donor or from a donor syngeneic with the latter.

Veto activity is defined as the capacity to specifically suppress cytotoxic T lymphocyte (CTL) precursors (CTL-p's) specific for veto-cell antigens (Muraoka S. and Miller R G., J Exp Med. 1980, 152:54-71; Claesson M H. and Miller R G., 1984, J Exp Med. 1984, 160:1702; Fink P J. et al., J. Immunol. 1984, 133:1775; Fink P J. et al., J. Immunol. 1984, 133:1769; Fink P J. et al., Annu Rev Immunol., 1988, 6:115; Sambhara S R. and Miller R G., Science, 1991, 252:1424; Thomas J M. et al., Transplantation 1995, 59:245)

Several types of veto cells or tolerance-inducing cells have been described (Lapidot T. et al., Blood 1992, 80:2406; Kaufman C L. et al., Blood 1994, 84:2436; Pierce G E. and Watts L M. Transplantation 1993, 55:882; Cobbold S P. et al., Nature 1986, 323:164; Kikuya S. et al., Proc Natl Acad. Sci. USA 1988, 85:4824; Kiyoshi H. et al., J Exp Med. 1992, 175:863; Pierce G E. and Watts L M., Transplant Proc. 1993, 25:331; Strober S. et al., J. Immunol. 1987, 138:699; Tscheming T. and Claesson M., Immunology Letters 1991, 29:223). It has been shown that some of the most potent veto cells are of T-cell origin and in particular a very strong veto activity has been documented for CD8+ CTL lines or clones (Cassell D J. and Forman J., J. Immunol. 1990, 144:4075; Claesson M H. and Ropke C., Curr Top Microbiol Immunol. 1986, 126:213; Fink P J. et al., J. Immunol. 1984, 133:1775; Annu Rev Immunol., 1988, 6:115; Tscherning T. and Claesson M H., Exp Clin Immunogenet. 1993, 10:179). In this modality, the suppression of effector CTL-p's directed against the veto cells is both antigen-specific and MHC-restricted. This suppression results from the unidirectional cytotoxicity of the veto cells to the responding CTLs and has been demonstrated to be mediated by apoptosis (Sambhara S R. and Miller R G., Science, 1991, 252:1424; Hiruma K. et al. J Exp Med. 1992, 175:863). The specificity of CTLs possessing veto activity was demonstrated by several studies to be unrelated to their T-cell receptor specificity (Sambhara S R. and Miller R G., Science, 1991, 252:1424; Claesson M H., Cell Immunol. 1987, 109:360; Sambhara S R. and Miller R G., J. Immunol. 1994, 152:1103).

Blocking experiments conducted with anti-CD8 or -MHC class I antibodies have suggested that elimination of responding CTL-p's by veto CTLs is induced via interaction of CD8 expressed on the latter with the α3 domain of MHC class I molecules expressed on the former (Sambhara S R. and Miller R G., Science 1991, 252:1424; Qi Y. et al., J Exp Med. 1996, 183:1973).

Blocking experiments conducted with anti-CD8 or -MHC class I antibodies have suggested that elimination of responding CTL-p's by veto CTLs is induced via interaction of CD8 expressed on the latter with the α3 domain of MHC class I molecules expressed on the former (Sambhara S R. and Miller R G., Science 1991, 252:1424; Qi Y. et al., J Exp Med. 1996, 183:1973)

In yet another approach to veto cell-mediated tolerance induction, non-alloreactive anti-third party CTLs were shown to be able to enhance allograft acceptance in mice (Reisner Y., Blood 1998, 92:265a; Bachar-Lustig E. et al., Blood 2000, 96:3739). However, the CTL preparation described in these studies may be contaminated with T-cells capable of inflicting GVHD and thus this method is not always suitable for human therapy.

It has also been demonstrated that hematopoietic progenitor cells (HPCs) are also endowed with potent veto activity (Rachamim N. et al., Transplantation 1998, 65:1386). The veto activity of HPCs may be at least partly mediated by production of immunosuppressive cytokines as demonstrated by studies in which cross-linking of CD8 on primate HPCs was shown toinduce TGF-β production and subsequent apoptosis of responder CTL-p's (Asiedu C. et al., Transplantation 1999, 67:372). Alternatively, murine studies have suggested that veto HPCs may induce apoptosis of CTL-p's via the Fas pathway (George J F. and Thomas J M., J Mol Med. 1999, 77:519).

The potential of employing dose escalation of HPCs to induce tolerance to MHC haploidentical, 3 loci-mismatched allografts in recipients has been demonstrated in human and murine studies. It has been observed in heavily conditioned human leukemia patients that administration of a sufficiently escalated dose of HPCs enabled durable engraftment of subsequent transplants from the same donor (U.S. Pat. No. 5,806,529 to Reisner et al. and Reisner Y. and Martelli M F., Curr Opin Immunol. 2000, 12:536). In the case of the murine studies, permanent acceptance of skin allografts in sublethally conditioned recipients was also achieved by using sufficiently high doses of enriched bone marrow (BM) HPCs which were adequately depleted of T cells and which were utilized in conjunction with other tolerance-inducing cells (Bachar-Lustig E. et al., Blood 1999, 94:3212). These results, therefore, indicate that escalation of administered doses of HPCs to sufficiently high levels may enable durable engraftment of MHC haploidentical, 3 loci-mismatched transplants in sublethally conditioned human recipients, as well.

This escalated HPC dose approach to tolerance induction, however, is not readily applicable for human transplantation since the number of HPCs required to induce durable recipient tolerance to MHC haploidentical, 3 loci-mismatched transplants in sublethally conditioned recipients is at least 3-fold higher than that which can be routinely harvested from human donors by state-of-the-art techniques.

Thus, although veto cell preparations and methods useful for inducing tolerance to transplants have been described in the prior art, such cell preparations suffer from inherent limitations which limits their applicability in human transplantation.

There is thus a widely recognized need for, and it would be highly advantageous to have, a method of inducing transplant tolerance which is easily applicable to human transplantation and yet be capable of inducing durable tolerance to transplanted organs, tissues or cells without risk of GVHD.

### SUMMARY OF THE INVENTION

According to one aspect of the present invention there is provided a method of inducing tolerance to a transplant transplanted from a donor to a recipient, the method comprising (a) culturing an HPC population under growth conditions suitable for inducing or enhancing veto activity in at least a portion of the HPC population, thereby generating a tolerance-inducing cell population; and (b) administering a dose of the tolerance-inducing cell population prior to, concomitantly with or following transplantation of the transplant, thereby inducing tolerance to the transplant in the recipient.

According to further features in preferred embodiments of the invention described below, the method of inducing tolerance to a transplant transplanted from a donor to a recipient further comprises the step of conditioning the recipient under sublethal, lethal or supralethal conditions prior to administering a dose of said tolerance-inducing cell population prior to, concomitantly with or following transplantation of the transplant, thereby inducing tolerance to the transplant in the recipient.

According to still further features in the described preferred embodiments, the donor is selected from the group consisting of an allogeneic donor and a xenogeneic donor.

According to still further features in the described preferred embodiments the donor and the recipient are both humans.

According to still further features in the described preferred embodiments the transplant is selected from the group consisting of cells, a tissue and an organ.

According to still further features in the described preferred embodiments the growth conditions are selected so as to induce myeloid differentiation in the HPC population.

According to still further features in the described preferred embodiments the growth conditions are selected so as to induce differentiation of CD33⁺ cells in the HPC population.

According to still further features in the described preferred embodiments the tolerance-inducing cell population predominantly displays a characteristic associated with a myeloid phenotype.

According to still further features in the described preferred embodiments the tolerance-inducing cell population predominantly expresses CD33.

According to still further features in the described preferred embodiments the veto activity is enhanced per cell in the HPC population.

According to still further features in the described preferred embodiments the dose of tolerance-inducing cells possesses sufficient veto activity so as to enable engraftment of MHC-mismatched transplants.

According to another aspect of the present invention there is provided a method of transplanting a transplant derived from a donor to a recipient, the method comprising (a) administering to the recipient a dose of cultured HPCs having enhanced veto activity as compared to non-cultured HPCs; and (b) transplanting the transplant to the recipient.

According to further features in the described preferred embodiments the method of transplanting a transplant derived from a donor to a recipient further comprises conditioning the recipient under sublethal, lethal or supralethal conditions prior to transplanting the transplant to the recipient.

According to still further features in the described preferred embodiments administering to the recipient a dose of cultured HPCs having enhanced veto activity as compared to non-cultured HPCs is performed prior to, concomitantly with or following transplanting the transplant to the recipient.

According to still further features in the described preferred embodiments the cultured HPCs are cultured in vitro.

According to still further features in the described preferred embodiments the cultured HPCs predominantly display a characteristic associated with a myeloid phenotype.

According to still further features in the described preferred embodiments the cultured HPCs predominantly express CD33.

According to still further features in the described preferred embodiments the enhanced veto activity is enhanced per cell in the cultured HPCs.

According to still further features in the described preferred embodiments the dose of cultured HPCs possesses sufficient veto activity so as to enable engraftment of MHC-mismatched transplants.

According to yet another aspect of the present invention there is provided a method of predicting the veto activity of a population of cultured HPCs, the method comprising (a) identifying cells displaying a characteristic associated with a myeloid phenotype in the population of cultured HPCs; and (b) determining within the population of cultured HPCs a ratio between cells displaying a characteristic associated with a myeloid phenotype and cells not displaying a characteristic associated with a myeloid phenotype.

According to further features in the described preferred embodiments identifying cells displaying a characteristic associated with a myeloid phenotype in the population of cultured HPCs is effected by detecting cells expressing a myeloid-specific molecule selected from the group consisting of an intracellular protein, a membrane-bound protein, a secreted protein, a messenger RNA (mRNA) transcript, a lipid, a carbohydrate, a hormone and a metabolite.

According to still further features in the described preferred embodiments the characteristic associated with a myeloid phenotype in the population of cultured HPCs is expression of CD33.

According to still further features in the described preferred embodiments identifying cells displaying a characteristic associated with a myeloid phenotype in the population of cultured HPCs is effected by a method selected from the group consisting of antibody recognition, ligand recognition and polymerase chain reaction (PCR) amplification.

According to still further features in the described preferred embodiments identifying cells displaying a characteristic associated with a myeloid phenotype in the population of cultured HPCs is effected by detection of a physical criterion selected from the group consisting of cellular morphology, cell size, cell density, cellular organelle morphology, cellular organelle size and cytoplasmic light scattering.

According to still further features in the described preferred embodiments identifying cells displaying a characteristic associated with a myeloid phenotype in the population of cultured HPCs is effected by histological staining or by a functional cellular or biochemical assay.

According to still further features in the described preferred embodiments predicting the veto activity of a population of cultured HPCs further comprises correlating the veto activity of the population of cultured HPCs with a ratio between cells displaying the characteristic associated with a myeloid phenotype and cells not displaying a characteristic associated with a myeloid phenotype.

According to an additional aspect of the present invention there is provided a method of isolating cells possessing veto activity from a population of cultured HPCs, the method comprising (a) contacting the population of cultured HPCs with a composition-of-matter capable of specifically binding to a cell displaying a characteristic associated with a myeloid phenotype; and (b) isolating the cells specifically contacting the aforementioned composition of matter.

According to further features in preferred embodiments the composition-of matter includes a binding moiety selected from the group consisting of an antibody, a T cell receptor (TCR), a biological ligand and a synthetic ligand.

According to still further features in the described preferred embodiments the composition-of-matter further includes a supporting matrix, whereas the binding moiety is attached to the supporting matrix.

According to still further features in the described preferred embodiments the composition-of-matter specifically binds to a molecule selected from the group consisting of a protein, a lipid and a carbohydrate.

According to still further features in the described preferred embodiments the composition-of-matter specifically binds to a cell displaying CD33.

According to yet an additional aspect of the present invention there is provided a method of treating or preventing an autoimmune disease in a subject, the method comprising administering to the subject a therapeutically effective amount of HPCs displaying at least one antigenic determinant associated with the autoimmune disease to thereby at least partially prevent or alleviate the autoimmune disease in the subject.

According to further features in preferred embodiments of the invention described below, the method of treating or preventing an autoimmune disease in a subject further comprises generating the HPCs displaying at least one antigenic determinant prior to the administering.

According to still further features in the described preferred embodiments, the generating is effected by pulsing a population of HPCs with a molecule including the at least one antigenic determinant.

According to still further features in the described preferred embodiments, the generating is effected by transforming a population of HPCs with at least one polynucleotide encoding the at least one antigenic determinant.

According to still further features in the described preferred embodiments, the population of HPCs is allogeneic with respect to the subject and whereas the polynucleotide further encodes an MHC molecule which is syngeneic with respect to the subject.

According to still further features in the described preferred embodiments, the method further comprising culturing the HPCs prior to, concomitantly with or following the generating.

According to still further features in the described preferred embodiments, the culturing is effected under conditions suitable for the formation of a myeloid phenotype in at least a portion of said HPCs.

According to still further features in the described preferred embodiments, the at least one antigenic determinant associated with the autoimmune disease is derived from a polypeptide selected from the group comprising myelin basic protein, insulin, glutamic acid decarboxylase and collagen.

According to still an additional aspect of the present invention there is provided a population of cells comprising HPCs displaying at least one antigenic determinant associated with an autoimmune disease.

According to further features in preferred embodiments of the invention described below, the HPCs are cultured HPCs predominantly displaying a characteristic associated with a myeloid phenotype.

According to still further features in the described preferred embodiments, the HPCs displaying at least one antigenic determinant are generated by pulsing the HPCs with a molecule including the at least one antigenic determinant.

According to still further features in the described preferred embodiments, the HPCs displaying at least one antigenic determinant are generated by transforming a the HPCs with at least one polynucleotide encoding the at least one antigenic determinant.

The present invention successfully addresses the shortcomings of the presently known configurations by providing a method of inducing tolerance to MHC-mismatched allografts in sublethally conditioned human recipients without risk of inflicting GVHD.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this re, no attempt is made to show details of the invention in more detail than is necessary for a fundamental understanding of the invention, the description taken with the drawings making apparent to those skilled in the art how the several forms of the invention may be embodied in practice.

In the drawings:

FIG. 1a is a histogram depicting the veto activity of CD34⁺ HPCs at different veto to effector cell ratios. Responder cells (10⁶ cells) and irradiated allogeneic stimulator cells (10⁶ cells) of the CD34⁺ HPC donor (solid bars) or a third party donor (hatched bars) were co-cultured for 5 days. Responder cells were then recultured for 7 days at limiting dilution. The Figure illustrates responder CTL activity as determined by ⁵¹Cr-release assay.

FIG. 1b is a histogram depicting the average responder CTL response in the presence (hatched bars) and absence (solid bars) of CD34⁺ HPCs, at a veto:responder cell ratio of 0.5. The veto effect was tested as in FIG. 1a and data was pooled from eleven independent experiments each using different donors.

FIG. 2 is a graph depicting the veto effect on the effector T cells upon removal of CD34⁺ HPCs at the end of the cell culture. Responder cells and irradiated allogeneic stimulators were co-cultured for 5 days with (solid circles) or without (open circles) the addition of CD34⁺ HPCs. The responder cells were then recultured with the original stimulators and IL-2 (10 U/ml) for 7 days. At the end of the reculture period the responders were isolated by E-rosetting with sheep erythrocytes and tested for CTL activity by ⁵¹Cr-release. A control experiment with (solid triangles) and without (open triangles) the addition of the same CD34⁺ HPCs to a mixed lymphocyte reaction (MLR) of the same responder cells against a third party, was carried out in parallel.

FIG. 3 is a histogram depicting the veto activity of CD34⁺ HPCs when added to cultures at different time points. Responder cells and irradiated allogeneic stimulator cells were co-cultured for 5 days with (solid bars) or without (hatched bars) addition of CD34⁺ HPCs. At the end of the culture period, responder cells were recultured at limiting dilution for 7 days and CTL activity was determined.

FIGS. 4a-b is a dot-plot depicting the levels of CD33 and CD34 surface expression in purified CD34⁺ HPCs following in vitro culture. CD34⁺ HPCs were cultured for 7 days in IMDM containing Flt-3 ligand (FL), stem cell factor (SCF) and thrombopoietin (TPO). Surface expression of CD34 and CD33 was analyzed by immunofluorescent flow cytometry before (FIG. 4a) and after (FIG. 4b) in vitro expansion. The percentage of each cell subpopulation is denoted in the relevant area of each dot plot.

FIGS. 5a-d is a dot-plot depicting the effect of in vitro-expanded CD34⁺ HPCs on expression of IFN-γ in responder T cells. Responder cells and irradiated allogeneic stimulator cells were co-cultured for 5 days in the absence (FIGS. 5a and c) and presence (FIGS. 5b and d) of cells obtained after a 12-day in vitro expansion of CD34⁺ HPCs. The stimulator cells in the MLR (veto:responder cell ratio of 0.125) were either from the donor of the CD34⁺ HPCs (FIGS. 5a and b) or from a third party (FIGS. 5c and d). After the 5-day MLR the cells were subjected to 7-day limiting dilution culture. The cells were then incubated with 8 ng/ml phorbol 12-myristate 13-acetate (PMA), 1 μM ionomycin and 2 μM monensin. Cells were then fixed and stained for detection of intracellular. IFN-γ. Percentages of each cell subpopulation are denoted in the relevant area of each dot plot.

### DESCRIPTION OF THE PREFERRED EMBODIMENTS

The present invention is of methods of inducing tolerance to a mismatched transplant of donor organs, tissues or cells in a recipient, methods of transplanting a transplant derived from a donor to a recipient, methods of predicting the veto activity of a population of cultured HPCs and methods of isolating cells possessing veto activity from a population of cultured HPCs. Specifically, the present invention relates to cultured HPCs possessing veto activity for use in transplantation, which cultured HPCs possess enhanced veto activity relative to non-cultured HPCs. As such, when administered to a recipient, such cultured HPCs facilitate engraftment of a mismatched transplant without risk of inflicting GVHD.

The principles and operation of the present invention may be better understood with reference to the drawings and accompanying descriptions.

Before explaining at least one embodiment of the invention in detail, it is to be understood that the invention is not limited in its application to the details set forth in the following description. The invention is capable of other embodiments or of being practiced or carried out in various ways. Also, it is to be understood that the phraseology and terminology employed herein is for the purpose of description and should not be regarded as limiting.

Various veto cell preparations capable of inducing graft tolerance have been described by the prior art.

For example, CD8⁺ CTL clones possessing veto activity and other T lymphocyte preparations specific for third party antigens were employed to prevent graft rejection. However, such T lymphocyte preparations are unsuitable for general use in tolerance induction due to the unacceptable rate of GVHD inflicted by the substantial fraction of residual alloreactive effectors contaminating such cell preparations.

Another more promising approach, which circumvents the aforementioned risk of inflicting GVHD, is based on the intrinsic veto activity of HPCs. As mentioned hereinabove, studies employing sublethally conditioned mice and heavily conditioned leukemia patients have shown that escalation of the numbers of administered HPCs to sufficiently high levels enables durable engraftment of MHC-haploidentical, 3 loci-mismatched transplants without inflicting GVHD. However, this approach cannot be currently applied to mismatched transplantations in sublethally conditioned humans since current state-of-the-art methods only make it possible to routinely harvest from a human donor approximately one-third of the HPCs required to attain this desirable goal.

Thus, all prior art approaches employing veto cells have failed to provide adequate solutions for inducing durable tolerance to such mismatched grafts without risk of inflicting GVHD.

While reducing the present invention to practice, cultured HPCs possessing up to 80-fold greater veto activity relative to non-cultured HPCs were generated under conditions non-permissive to survival of CTLs. Hence, the method according to the present invention enables the generation of a veto cell preparation possessing up to 80-fold more total veto activity than that possessed by the total number of primary non-cultured HPCs which can be harvested from a human donor employing state-of-the-art techniques, while being free of contaminating alloreactive CTLs.

Thus, the method of the present invention can be used to confer tolerance to xenografts or MHC mismatched or matched (minor disparities) allografts in sublethally conditioned human recipients while avoiding the risk associated with GVHD.

Thus, according to one aspect of the present invention there is provided a method of inducing tolerance to a transplant transplanted from a donor to a recipient. The method according to the present invention can be used to induce tolerance to a transplant of organs, appendages, tissues or cells used to replace defective or missing homologues or to be used in an adoptive therapy context.

The method according to the present invention can be applied to induce tolerance to a transplant which originates from the same species as the recipient (allogeneic) or a transplant which originates from a different species (xenogeneic).

Preferably, the method of the present invention is employed to induce tolerance to a transplant transplanted from a human donor to a human recipient (allogeneic transplantation).

Examples of organ transplants include, but are not limited to, kidney, heart, pancreas, lung and liver.

Examples of appendage transplants include, but are not limited to, arms, legs, hands, feet, fingers, toes and portions thereof.

Examples of tissue transplants include, but are not limited to, dermal, pancreatic and nerve tissues.

Examples of cell transplants include, but are not limited to, HPCs and embryonic stem cells. Transplants of HPCs derived from BM, mobilized peripheral blood (by, for example, leukapheresis), fetal liver, yolk sac and cord blood can be employed, for example, to treat hematological deficiencies, including those arising as a consequence of medical treatment. Such hematological deficiencies can be, but are not limited to, leukemias, such as acute lymphoblastic leukemia (ALL), acute nonlymphoblastic leukemia (ANLL), acute myelocytic leukemia (AML) or chronic myelocytic leukemia (CML). Other such hematological deficiencies can be, but are not limited to, severe combined immunodeficiency (SCID) syndromes (such as, for example adenosine deaminase (ADA) deficiency and X-linked SCID (XSCID)), osteopetrosis, aplastic anemia, Gaucher's disease, thalassemia and other congenital or genetically-determined hematopoietic abnormalities.

In addition to applicability in the context of replacement transplantation described above, the method according to the present invention can be used to induce tolerance to organs, tissues or cells transplanted in the context of adoptive therapy. Conditions for which this therapeutic modality is applicable include, but are not limited to, malignant, viral, autoimmune and parasitic diseases. Such adoptive cell therapies can be employed, for example, towards treatment of cancer or acquired immunodeficiency syndrome (AIDS) via transplantation of donor-derived immune effectors, such as T lymphocytes or natural killer (NK) cells directed, either naturally or due to genetic modification, against cells expressing tumor-associated or human immunodeficiency virus (HIV) antigens, respectively.

According to the present invention, the method of inducing tolerance to a transplant from a donor to a recipient is effected by culturing an HPC population under growth conditions suitable for inducing or enhancing veto activity in at least a portion of the cultured HPC population, thereby generating a tolerance-inducing cell population.

As described in detail in the Examples section below, culturing of HPCs according to the method of the present invention generates a cell population possessing enhanced veto activity both per cell and per total population as a result of expansion in cell numbers and differentiation of cells. As previously mentioned above, this can lead to an 80-fold increase in the total veto activity of populations of cultured HPCs relative to non-cultured HPCs. Hence, HPCs cultured according to the method of the present invention are highly suitable for induction of tolerance to haploidentical, 3 loci-mismatched allografts in sublethally conditioned human recipients since the veto activity thereof is substantially higher than that required.

Due to this enhanced veto activity, the method of the present invention can employ relatively small doses of cultured HPCs to induce tolerance to MHC mismatched allografts in sublethally conditioned recipients.

According to a preferred embodiment of the present invention, the factors employed to culture HPCs direct the differentiation and expansion of cells displaying characteristics associated with a myeloid phenotype. Culturing of HPCs with such factors is described in detail in the Examples section, hereinunder.

Since it is known that non-cultured HPCs are essentially free of alloreactive CTLs and hence do not inflict GVHD, culturing of HPCs according to the present invention is effected in the presence of factors inducing myeloid differentiation and in the absence of cytokines such as IL-2, TNF-α and IFN-γ or of any form of antigenic stimulation. This ensures that any contaminating T lymphocytes, being non-stimulated, will not survive the culture conditions and thus will not contaminate the cultured HPC preparation utilized by the method of the present invention.

Following generation of the cultured HPCs, the method of the present invention is further effected by administering a dose of the HPCs prior to, concomitantly with or following transplantation of the transplant, thereby inducing tolerance to the transplant in the recipient. In order to achieve optimal tolerance induction, doses of cultured HPCs can be administered, for example, one or more times during any combination of the periods prior to, concomitant with or following transplantation of the transplant.

Preferably, cultured HPCs are administered concomitantly with transplantation of the transplant since, as described in detail in the Examples section below, veto effect is optimal when cultured HPCs are present at the time of exposure of responding CTLs to allogeneic stimulator cells.

Preferably, the method of inducing tolerance to a transplant from a donor to a recipient according to the teachings of the present invention, also includes an additional step in which the recipient is conditioned under sublethal, lethal or supralethal conditions prior to transplantation.

Such conditioning is dependent on the nature of the transplant and the condition of the recipient. The recipient may be conditioned under sublethal, lethal or supralethal conditions, for example, by total body irradiation (TBI) and/or by treatment with myeloablative and immunosuppressive agents according to standard protocols. For example, a sublethal dose of irradiation is within the range of 1-7.5 Gy. TBI, a lethal dose is within the range of 7.5-9.5 Gy TBI and a supralethal dose is within the range of 9.5-16.5 Gy TBI. Examples of myeloablative agents include busulphan, dimethyl mileran, melphalan and thiotepa and examples of immunosuppressive agents include prednisone, methyl prednisolone, azathioprine, cyclosporine A, cyclophosphamide, fludarabin, etc.

In a preferred embodiment of the present invention the recipient is conditioned sublethally.

Prior to administration of cultured HPCs for induction of tolerance to a transplant in a recipient, the preparation of cultured HPCs should preferably be monitored to ascertain whether it possesses sufficient veto activity to induce the required level of transplant tolerance.

In addition, it may be necessary to compare the veto activity possessed by different cultured HPC preparations in order to select those possessing the optimal veto activity required for induction of transplant tolerance.

Thus, according to a further aspect of the present invention, there is provided a method of predicting the veto activity of a population of cultured HPCs.

According to this aspect of the present invention, the method of predicting the veto activity of a population of cultured HPCs is effected by identifying cells displaying a characteristic associated with a myeloid phenotype in the population of cultured HPCs.

For example, such a characteristic can be a myeloid-specific molecule such as a protein, a messenger RNA (mRNA) transcript, a lipid, a carbohydrate, a hormone or a metabolite. Examples of typical pan-myeloid surface markers include CD33 and CD13.

Methods employed to detect cells expressing or displaying molecules whose expression is associated with a myeloid phenotype can be based on antibody- or ligand-directed specific binding reagents.

In the case of membrane or cytoplasmic molecules, fluorescent flow cytometry or microscopic analysis can be employed. Detection of relevant secreted molecules can be performed, for example, via enzyme-linked immunosorbent assay (ELISA). Alternatively, methods based on reverse-transcriptase polymerase chain reaction (RT-PCR) can be employed to detect mRNA transcription of genes associated with a myeloid phenotype. Also, immunohistochemical stains such as, for example, eosin, hematoxylin, nitroblue tetrazolium or Fast-Green/Neutral-Red can be employed to detect cells of various myeloid lineages.

According to another preferred embodiment of the present invention, detection of molecules associated with a myeloid phenotype is performed via fluorescent flow cytometric analysis. As shown in the Examples section below, the majority of cells within HPC-derived cell preparations possessing enhanced veto activity are shown, via immunofluorescent flow cytometry to express the pan-myeloid marker CD33.

Alternative methods of detecting cells displaying characteristics associated with a myeloid phenotype within a population of cultured HPCs can be based on optically analyzing the size, shape and/or cytoplasmic granularity of cells in such a population. For example, cells displaying a size (forward scatter), granularity (side scatter), shape or a combination of parameters characteristic of a myeloid phenotype can be detected by flow cytometry.

Following identification of cells displaying characteristics associated with a myeloid phenotype, the veto activity of a population of cultured HPCs is predicted by determining a ratio between cells displaying a characteristic associated with a myeloid phenotype and cells not displaying a characteristic associated with a myeloid phenotype within the population.

As is further described in the example section which follows, the cultured HPCs of the present invention are characterized by a ratio of approximately 7:1 myeloid to non-myeloid cells.

Although such a ratio is preferred, lower ratios of even 1:9 (myeloid/non-myeloid) or less are also acceptable providing the actual number of myeloid cells present in the culture is sufficient for inhibiting CTL activity in the recipient thereby inducing tolerance.

According to a further aspect of the present invention there is provided a method of isolating cells possessing veto activity from a population of cultured HPCs.

According to the present invention, the method of isolating cells possessing veto activity from a population of cultured HPCs is effected by contacting the latter with a composition-of-matter capable of specifically binding to a cell displaying a characteristic associated with a myeloid phenotype. Such compositions-of-matter can include recognition moieties derived from antibodies, TCRs, biological ligands or synthetic ligands and may include a supporting matrix, such matrix being specific to the subsequent step, according to the method of the present invention, of isolating contacted cells. Examples of such supporting matrices and isolation methods in which they are employed include agarose beads for affinity purification columns, plastics for culture flask-panning purification methods or metal-particles for magnetic cell sorting methods.

Following contacting cultured HPCs with a composition-of-matter capable of specifically binding to a cell displaying a characteristic associated with a myeloid phenotype, isolation of cells possessing veto activity from a population of cultured HPCs is further effected by isolating cells specifically contacting the aforementioned composition of matter. This can be performed via the methods alluded to in the previous step of this method. As discussed above regarding methods of detecting cells displaying a characteristic associated with a myeloid phenotype, isolation of such cells can also be performed via flow cytometry.

A general alternative to the aforementioned methods of isolating cells possessing enhanced veto activity is to deplete cells not expressing a molecule associated with a myeloid phenotype from HPC-derived cell cultures in a manner analogous to those elaborated above for isolating cells expressing a molecule associated with a myeloid phenotype. Depletion of non-veto activity enhanced cells from within a cell population can also be effected via complement-mediated cell lysis of cells specifically expressing surface molecules not expressed on cells displaying characteristics associated with a myeloid phenotype.

Finally, yet another method of isolating cells possessing veto activity from a population of cultured HPCs can be based on differences in physical properties between cells displaying characteristics associated with a myeloid phenotype and cells which do not. This can be effected, for example, via centrifugation through a density gradient capable of specifically isolating cells possessing a density characteristic of a myeloid phenotype.

In addition to the above, HPCs (preferably cultured as described hereinabove) can also be utilized to treat an individual suffering from an autoimmune disease including, but not limited to, multiple sclerosis, rheumatoid arthritis, insulin-dependent (Type I) diabetes, Grave's disease, Crohn's disease, systemic lupus erythematosus, myasthenia gravis, thyroiditis, thrombocytopenic purpura, chronic glomerulonephritis, atherosclerosis and autoimmune bullous skin diseases such as pemphigus vulgaris, bullous pemphigoid, pemphigus foliaceus or any other disease involving autoreactive T cells.

The cause and/or maintenance of such diseases is believed to be at least partly mediated by autoreactive T cells specific for MHC-restricted peptides derived from proteins expressed in affected tissues.

For example, autoreactive T cells directed against myelin basic protein expressed in nerve tissue contribute to the formation of multiple sclerosis, autoreactive T cells directed against collagen expressed in the joints contribute to the formation of rheumatoid arthritis and autoreactive T cells directed against glutamic acid decarboxylase and insulin expressed in the pancreas contribute to the formation of type I diabetes.

Thus, suppression or elimination of autoreactive T cells associated with autoimmune reactions can be used to alleviate or prevent such autoimmune diseases.

Thus, according to yet another aspect of the present invention, there is provided a method for treating an autoimmune disease in a subject.

The method according to this aspect of the present invention is effected by administering to the subject HPCs displaying one or more antigenic determinants associated with the autoimmune disease.

The antigenic determinants form a part of a molecule or molecules which are introduced into or onto the HPCs in a manner which, in the case of peptides, enables association with MHC and thus MHC-restricted surface display.

The display of such peptides at the cell surface can be effected via peptide pulsing or by incubation of cells with exogenous peptides (Day PM et al., Proc Natl Acad. Sci. USA. 1997, 94:8064) or by transforming HPCs with a polynucleotide encoding such a peptide.

In any case, the pulsed or expressed peptides are designed such that MHC-restricted display is enabled.

Examples of molecules containing antigenic determinants which can be used by this aspect of the present invention include peptides derived from proteins such as, myelin basic protein, collagen, glutamic acid decarboxylase or insulin; MHC molecules, MHC subunits, MHC-peptide complexes or MHC-peptide single-chain fusion proteins; anti-TCR idiotype antibodies or portions thereof, anti-TCR idiotype TCRs or portions thereof or biological TCR idiotype ligands. Other examples of molecules containing antigenic determinants include synthetic TCR idiotype ligands.

The method according to this aspect of the present invention can utilize either allogeneic HPCs in which case MHC antigens are preferably also expressed and/or displayed by the allogeneic HPCs, or syngeneic HPCs which express or display peptides in association with endogenous MHC antigens.

The HPCs employed to treat an autoimmune disease are preferably cultured as described hereinabove in order to enhance veto activity thus greatly facilitating the suppression or elimination of autoreactive T cells. Such culturing can be effected prior to or following genetic transformation or prior to peptide pulsing.

It will be appreciated, however, that the use of non-cultured HPCs derived for example, from the subject or a syngeneic, allogeneic or xenogeneic donor for suppressing or eliminating autoreactive T cells is also envisaged by the present invention.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D. and Higgins S. J., eds. (1985); “Transcription and Translation” Hames, B. D. and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Enhancement of Veto Activity in Cultured. HPCs Relative to Non-Cultured HPCs

Freshly isolated, non-cultured HPCs have been demonstrated to possess veto activity and were shown, in sublethally irradiated mice and in heavily conditioned leukemia patients, to induce durable engraftment of MHC-haploidentical, 3 loci-mismatched transplants without inflicting GVHD when administered in sufficiently high numbers, i.e. at megadoses. As discussed hereinabove, it has been determined, however, that application of this method to sublethally conditioned human transplant recipients requires administration of at least three times more HPCs than the number which can be harvested from donors by state-of-the-art techniques. Therefore, as described in this example, a method was developed to augment the veto activity of human HPCs to sufficiently high levels to enable use thereof in inducing transplant tolerance in sublethally conditioned human transplant recipients.

In addition, this study examines mechanisms responsible for the veto activity mediated by cultured HPCs and their resultant phenotype following differentiation in culture.

Materials and Methods:

Peripheral blood progenitor cell (PBPC) collection, processing and CD34⁺ HPC purification: PBPC were collected from healthy unrelated allogeneic donors following mobilization with standard doses of recombinant human G-CSF (rhG-CSF). To prepare mononuclear cells from PBPC, cells were placed on a gradient of Ficoll-paque Plus (Amersham Pharmacia Biotech AB, Uppsala, Sweden) and centrifuged. CD34⁺ HPCs were purified by magnetic cell sorting, using magnetic beads linked to anti-CD34 mAb (Miltenyi Biotec, Bergisch Gladbach, Germany) and purity was analyzed by flow cytometry.

MLR: Responder lymphocytes from donor C were reacted with stimulator cells from two donors (A and B) previously selected by class I HLA typing to be non-cross reactive with each other. Cells from donor C (10⁶ cells/ml) were cultured with irradiated (30 Gy) stimulator cells (10⁶ cells/ml), with or without addition of CD34⁺ HPCs (0.5×10⁶ cells/ml) from donor A. The cells were cultured in 6 ml of complete tissue culture medium (CTCM)+10% fetal calf serum (FCS, Biological. Industries, Kibbutz Beit Haemek, Israel) for 5 days. CTCM is RPMI 1640 containing 2 mM L-glutamine, 100 U/ml penicillin, 0.1 mg/ml streptomycin, 2 mM HEPES, 1 mM sodium pyruvate, 0.1 mM non-essential amino acids and 5×10⁵M 2-mercaptoethanol (Biological Industries, Kibbutz Beit Haemek, Israel).

Limiting dilution culture: Cells were harvested from the MLR culture and separated by centrifugation on a cushion of Ficoll-Paque. Serial dilutions of responder cells (from 4×10⁴ to 0.2×10³ cells/well) were then seeded in round-bottom 96-well plates at 16 replicates per dilution. Each well contained 10⁵ irradiated stimulator cells from the original donor used in the bulk MLR. The cultures were incubated for 7 days in CTCM+10% FCS and 10 U/ml recombinant human IL-2 (EuroCetus, Amsterdam, The Netherlands) in a final volume of 0.2 ml.

Estimate of CTL activity: Cytotoxic activity was assayed by transferring 100 μl of limiting dilution cultures to conical-bottom 96-well plates (Greiner, Frickenhausen, Germany) and incubating for 4 hr the effector cells with 5×10^{3 51}Cr-labeled concanavalin A (Sigma, St. Louis) stimulated blasts (target cells) from the donor or from a third party. The mean radioactivity of 16 replicate samples was calculated and percent specific lysis was determined by the following equation: 100×(experimental release−spontaneous release)/(total release−spontaneous release). The release of ⁵¹Cr by target cells, either cultured in medium alone or lysed with 1% SDS, was defined as spontaneous or total release, respectively.

Frequency calculation of CTL-p's: To calculate the frequency of CTL-p's from the limiting dilution culture readout, we used the equation: ln y=−fx+ln a, which represents the zero-order term of the Poisson distribution (Taswell C. et al., J Exp Med. 1980, 15:1372) where Y is the percentage of nonresponding cultures, x is the number of responding cells per culture, f is the frequency of responding precursors and a is the y-intercept theoretically equal to 100%. Microwell cultures were considered positive for cytolytic response when they exceeded the mean spontaneous release value by at least three standard deviations of the mean. The CTL-p frequency, was determined from the slope of the resultant line drawn utilizing linear regression analysis of the data.

The role of cell contact in mediation of veto effect (transwell assay): Responder and irradiated stimulator cells (10⁶ cells/ml) were placed in the lower chamber of a transwell culture system (Transwell-col, Costar Corp., Cambridge, Mass.) and purified donor CD34⁺ HPCs (0.5×10⁶ cells/ml) were placed together with responder cells (1×10⁶ cells/ml) in the upper chamber. After 5 days of incubation, the cells were harvested and recultured at limiting dilution.

In vitro culture of HPCs: Purified CD34⁺ HPCs (10⁵ cells in 1 ml) were cultured in 24-well plates in Iscove's Modified Dulbecco's Medium containing 10% FCS, 2 mM L-glutamine, 100 U/ml penicillin, 0.1 mg/ml streptomycin and 50 ng/ml recombinant human Flt3-ligand (FL), 50 ng/ml recombinant human stem cell factor (SCF) and 1 ng/ml recombinant human thrombopoietin (TPO) (purchased from R&D Systems, Minneapolis, Minn.). On day 5, the same doses of FL, SCF and TPO were added and on day 7-12 cells were harvested and tested for veto activity.

Flow cytometry analysis of intracellular IFN-γ content: Cells from 5-day MLR and additional 7-day limiting dilution culture were harvested and activated (10⁶ cells/ml) with 8 ng/ml phorbol 12-myristate 13-acetate (PMA, Sigma, St. Louis), 1 μM ionomycin (Sigma, St. Louis) and 2 μM monensin (Sigma, St. Louis). Monensin was added to the cultures to cause intracellular accumulation of newly synthesized proteins by arresting transport in the Golgi complex. After a 2 hr incubation at 37° C., cells were collected, washed in PBS and aliquoted at ˜0.2×10⁶ cells/tube for immunofluorescent staining. Cell suspensions were supplemented with 50 μg chrompure human IgG (Jackson, West Grove, Pa.) to reduce nonspecific staining and then stained with anti-CD3-Cy-Chrom (Pharmingen, San Diego, Calif.). Cells were then washed, fixed and permeabilized with fixation and permeabilization reagents (Leucoperm kit, Serotec, Oxford, U.K) according to manufacturer's instructions. For intracellular staining, cells were incubated with anti-IFN-γ-FITC (Pharmingen, San Diego, Calif.) or matching isotype control reagent. Samples were analyzed using a FACScan flow cytometer and data were analyzed using LYSIS II software (Becton Dickinson, San Jose).

Experimental Results:

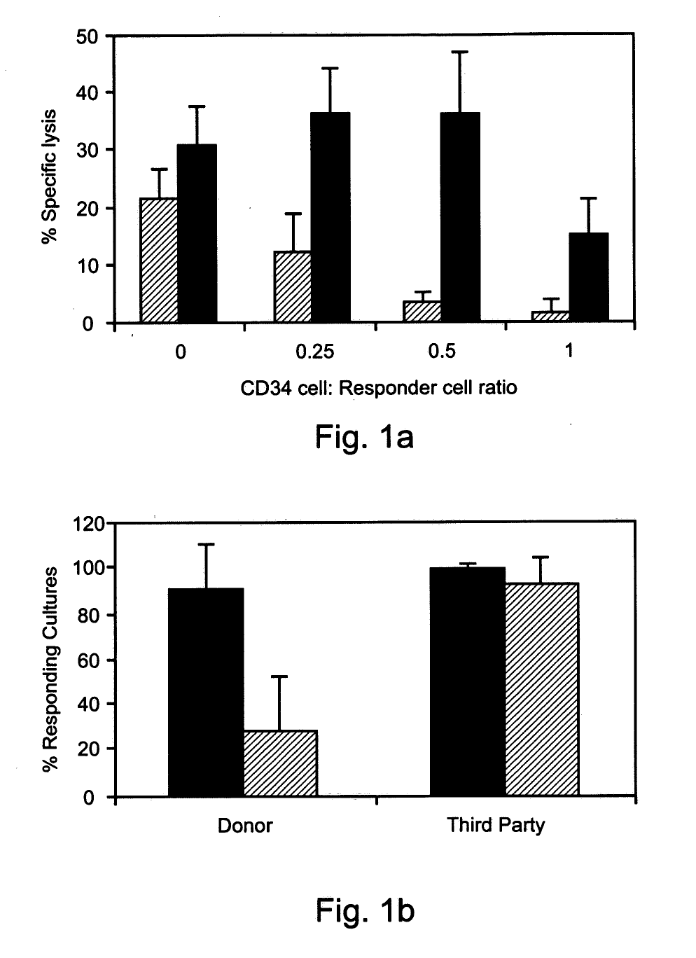

The veto activity of non-cultured CD34⁺ HPCs: In order to establish a baseline for determining whether culture of CD34⁺ HPCs increases veto activity we first characterized the veto activity of non-cultured CD34⁺ HPCs at different CD34⁺ HPC to responder cell ratios, so as to define the ratio at which optimal veto activity is exhibited. As can be seen in FIG. 1a, the optimal veto activity was displayed at a ratio of 0.5:1 CD34⁺ HPCs to responder cells. At this ratio, 80% inhibition of CTL activity was recorded when the culture contained stimulator cells of the CD34⁺ HPC donor, while no inhibition was detected when using stimulators of third party origin. Based on this dose response curve, all subsequent experiments were carried out at a CD34⁺ HPC to responder cell ratio of 0.5. FIG. 1b illustrates that non-cultured CD34⁺ HPCs display veto activity with respect to syngeneic but not third party stimulators. Data shown represents average veto activity of CD34+ HPCs derived from 11 independent experiments performed on cells from separate individuals.

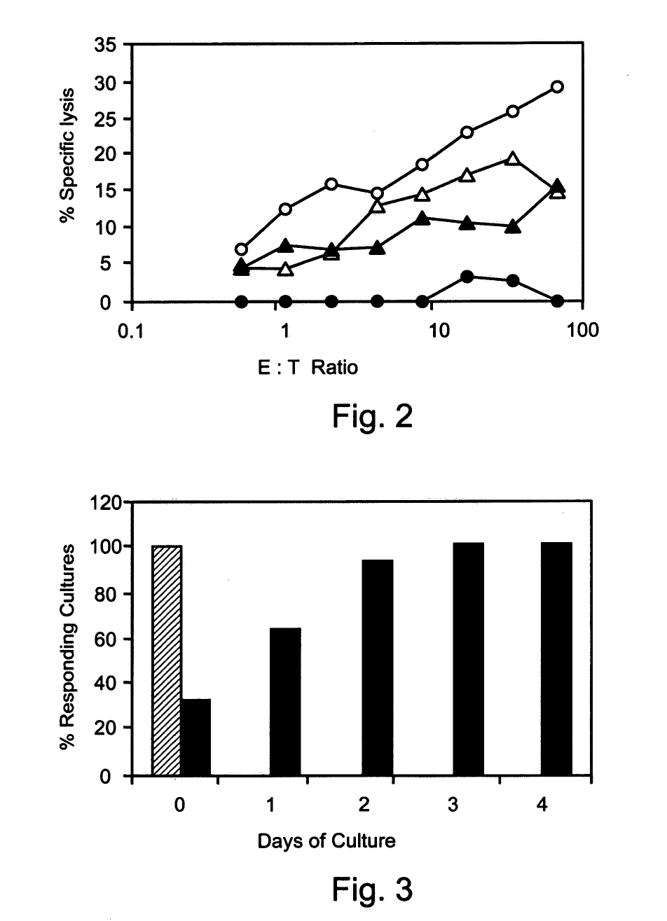

The veto activity of CD34⁺ HPCs is not due to cold target inhibition: It could be argued that the responder CTL suppression observed in the presence of CD34⁺ HPCs is due to cold target inhibition. Namely, that the CD34⁺ HPCs compete at the end of the culture period with the ⁵¹Cr-labeled target cells for CTL mediated lysis. Such competition should only occur when the CD34⁺ HPCs and the target cells are syngeneic for HLA class I antigens and, in such case, chromium release should be reduced in anti-donor cultures but not in anti-third party cultures. To eliminate this possibility, we isolated the effector T cells at the end of the bulk culture period by E-rosetting with sheep erythrocytes. The CTL activity of this purified T cell fraction, shown by flow cytometry to be >90% pure and free of CD34⁺ HPCs, was tested. As can be seen in FIG. 2, the veto effect is retained after removal of CD34⁺ HPCs, hence the veto activity of CD34⁺ HPCs is not due to cold target inhibition.

Inhibition of anti-donor response is optimal when CD34⁺ HPCs are added prior to 24 h of culture. In order to determine the kinetics of veto activity by CD34⁺ HPCs, the latter were added at different time points after initiation of culture. As can be seen in FIG. 3, optimal inhibition of the response occurs prior to day 1 of culture. Inhibition was no longer detectable when CD34⁺ HPCs were added from day 2 onward. Thus, the veto effect of CD34⁺ HPCs, similarly to other veto cells acts at the early induction phase of alloreactive CTLs (Muraoka S. et al., Eur J. Immunol. 1984, 14:1010; Uberti J. et al., Blood 1992, 1:261; Thomas J M. et al., Transplantation 1994, 57:101; Reich-Zeliger S. et al., Immunity 2000, 13:507).

The veto effect of CD34⁺ HPCs is independent of alloreactivity: In order 7 to eliminate the possibility that reduction of . . . anti-donor reactivity observed in the presence of CD34⁺ HPCs is mediated by contaminating cells displaying alloreactivity against the responder effector cells, we compared the veto effect of CD34⁺ HPCs to that exhibited by the CD34⁻ fraction which contains a significant fraction of T cells. As shown in Table 1 below, while a marked reduction of CTL-p frequency was found in MLRs to which purified CD34⁺ HPCs were added, no inhibition was detected when the CD34⁻ fraction was added. Thus, the veto activity of cells within the CD34⁺ HPC fraction cannot be attributed to contaminating alloreactive CD34⁻ cells.

TABLE 1
The veto effect is independent of alloreactivity^{a}, and
requires cell contact^{b}

| | MLR against A | | MLR against B | |
|---|---|---|---|---|
| | CTL | | CTL activity | |
| | activity^{d} | | (% | Veto activity |
| | (% Responding | Veto activity^{e} | Responding | (% |
| Cells^{c} | cultures) | (% Inhibition) | cultures) | Inhibition) |
| None | 87.5 | | 93.75 | |
| CD34⁺ | 18.75 | 79 | 62.5 | 33 |
| CD34⁻ | 100 | 0 | ND | |
| CD34⁺ in | 87.5 | 0 | 100 | 0 |
| transwell^{d} | | | | |

^{a}The role of alloreactivity was evaluated by testing the veto effect of the CD34⁺ HPCs in comparison to the CD34⁻ cells, which include a significant fraction of alloreactive T cells compared to the former.
^{b}The role of cell contact was evaluated via a transwell assay. Briefly, the responder cells to be tested for their CTL response were cultured with the irradiated stimulator cells in the lower chamber of the transwell plate. The CD34⁺ HPCs were cultured together with responder cells in the upper chamber.
^{c}Cells from donor A were added to a 5-day MLR against stimulator cells from donor A or B.
^{d}The CTL activity was tested at the end of 7-day limiting dilution culture in microtiter plates. Wells were scored positive for CTL activity when ⁵¹Cr-release exceeded the mean spontaneous release value by at least three standard deviations of the mean. Data shows percent responding cultures at a cell concentration of 10⁴ responder cells/well.
^{e}Veto activity of tested cells was evaluated by their capacity to inhibit alloreactive CTL-p's in the MLR to which they were added at 0.5:1 veto:responder cell ratio.

The veto effect of CD34⁺ HPCs requires cell contact: To study whether the veto activity of CD34⁺ HPCs can be mediated by soluble factors, MLRs were performed in transwell plates composed of two chambers separated by a membrane. The responder cells and the irradiated donor stimulator cells were placed in the lower chamber and the purified CD34⁺ HPCs were placed together with the responder cells in the upper chamber. As can be seen in Table 1 the CTL-p frequency was not reduced when the CD34⁺ HPCs were separated from the stimulated responder cells by a membrane allowing passage of soluble factors but not cells. Therefore, CD34⁺ HPC-mediated veto activity requires cell to cell contact between the veto cells and the inhibited responder cells.

Expansion of veto cells following culture of CD34⁺ HPCs: Culture of CD34⁺ HPCs in vitro is usually associated with significant loss of self-renewal capacity and with differentiation of cells displaying characteristics associated with a myeloid phenotype. To test whether CD34⁺ HPCs can be cultured in vitro without loss of veto activity, CD34⁺ HPCs were cultured for 7-12 days in the presence of an early-acting cytokine cocktail including FL, SCF and TPO (Qiu L. et al., J Hematother Stem Cell Res. 1999, 8:609) at concentrations of 50, 50 and 1 ng/ml, respectively.

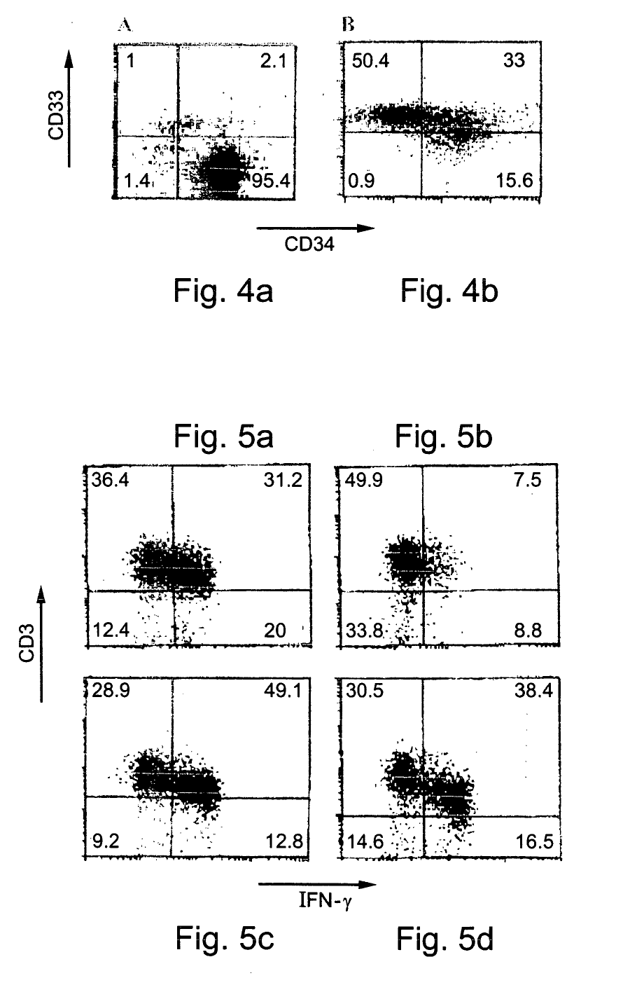

Immunofluorescence phenotyping revealed that, while prior to culture about 97% of the cells were CD34⁺ CD33⁻ (FIG. 4a), the expanded cells comprised three major subpopulations; CD34⁺ CD33⁻ (15.6%), CD34⁺ CD33⁺ (33.0%) and CD34⁻CD33⁺ (50.5%) (FIG. 4b). Furthermore, the majority of the cells were CD13⁺ (79%) and CD4^{low} (80%) and did not express CD8, CD20 or CD56 (data not shown). Thus, as expected, these culture conditions yielded cells having differentiated predominantly along the myeloid lineage.

The veto activity of these in vitro-cultured: cells was characterized via their effect on CTL-p frequency in bulk cultures. As shown in Table 2 below, the expanded cells harvested after 7 to 10 days of culture markedly inhibited anti-donor CTL response (79-100% inhibition). In contrast, the anti-third party CTL response was notably less inhibited (0-47% inhibition). Significant veto activity was exhibited by the expanded cells at veto:responder cell ratios of 0.5, 0.25 and 0.125 (Table 2). Thus, compared to the initial CD34⁺CD33⁻ cell fraction in which threshold veto activity was detected at a veto:responder cell ratio of 0.5, the veto activity of the expanded cells was increased 4-fold. Furthermore, the 7- to 20-fold expansion in cell numbers observed by the end of culture yielded an effective 28- to 80-fold total increase in veto activity relative to non-cultured CD34⁺ HPCs.

TABLE 2
Veto activity of in vitro cultured CD34⁺ HPCs: determination of veto
activity at different veto:responder cell ratios

| | CD34⁺ HPC | | | | | |
|---|---|---|---|---|---|---|
| | veto Cells^{a} | | MLR against A | | MLR against B | |
| | (veto:responder | | CTL | Veto | CTL | Veto |
| | ratio) | | activity^{b} | activity^{c} | activity | activity |
| | Before | After | (% Resp | (% | (% Resp | (% |
| Expts | culture | culture | cultures) | Inhibition) | cultures) | Inhibition) |
| 1 | — | — | 87.5 | | 93.75 | |
| | 0.5 | — | 18.75 | 79 | 62.5 | 33 |
| | — | 0.5 | 6.25 | 93 | 50 | 47 |
| | — | 0.25 | 18.75 | 79 | 62.5 | 33 |
| 2 | — | — | 62.5 | | 100 | |
| | — | 0.25 | 6.25 | 90 | 100 | 0 |
| | — | 0.125 | 0 | 100 | 100 | 0 |

^{a}Responder cells and irradiated stimulator cells (from donor A or B) were cocultured for 5 days in the absence or presence of CD34⁺ HPCs from donor A, obtained before and after culture (for 7 or 10 days in Experiment 1 or 2, respectively). The responder cells were then recultured for 7 days at limiting dilution in microtiter plates.
^{b}Wells were scored positive for CTL activity when ⁵¹Cr-release exceeded the mean spontaneous release value by at least three standard deviations of the mean. Data shows percent responding cultures in Experiments 1 and 2, respectively, at a cell concentration of 1 × 10⁴ and 4 × 10⁴ responder cells/well, respectively.
^{c}Veto activity of tested cells was evaluated by their capacity to inhibit alloreactive CTL-p's in the MLR to which they were added at the indicated veto:responder cell ratios.

Finally, the specific inhibitory effect of the expanded cells was also revealed by their effect on levels of intracellular IFN-γ content in responding T cells (FIGS. 5a-d). The percentage of CD33⁺ cells displaying intracellular IFN-γ following stimulation by cells of the CD34⁺ HPC donor, when tested at a 0.125:1 veto:responder cell ratio in the absence of added cells, was 31.2% and it was reduced to 7.5% upon addition of in vitro cultured HPCs. In contrast, when stimulated by third party cells, the fraction of CD3⁺IFN-γ⁺ cells was 49.1% in the absence of added cells and 38.4% upon addition of the cultured HPCs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of inducing tolerance to a transplant transplanted from a donor to a recipient, the method comprising:
(a) culturing an HPC population derived from the donor under growth conditions suitable for generating a cultured HPC population having a tolerance-inducing activity, wherein cells displaying a characteristic associated with a myeloid phenotype make up at least 83.5 percent of said cultured HPC population; and
(b) administering to the recipient a dose of said cultured HPC population prior to, concomitantly with or following transplantation of the transplant, wherein the donor is allogeneic or xenogeneic with the recipient, thereby inducing tolerance to the transplant in the recipient.

2. The method of claim 1, wherein said dose of said cultured HPC population is at least 20 times lower than a dose of said HPC population derived from the donor which is capable of inducing said tolerance.

3. The method of claim 1, further comprising the step of conditioning the recipient under sublethal, lethal or supralethal conditions prior to step (b).

4. The method of claim 1, wherein the transplant is selected from the group consisting of cells, a tissue and an organ.

5. The method of claim 1, wherein said growth conditions are selected so as to induce differentiation into CD33+ cells in said HPC population derived from the donor.

6. The method of claim 1, wherein said cultured HPC population is predominantly CD33+.

7. The method of claim 1, wherein said tolerance-inducing activity is enhanced per cell in said cultured HPC population relative to said HPC population derived from the donor.

8. The method of claim 1, wherein the transplant is MHC-mismatched with the recipient, and whereas said dose of said cultured HPC population possesses sufficient tolerance-inducing activity so as to enable engraftment of the transplant.

9. The method of claim 1, wherein said culturing is effected in absence of exogenously added IL-2, TNF-α and/or IFN-γ.

10. The method of claim 1, wherein the recipient and/or the donor is a human.

11. The method of claim 1, wherein said HPC population derived from the donor is a population of purified CD34+ cells.

12. The method of claim 1, wherein said characteristic associated with a myeloid phenotype is surface expression of CD33.

13. The method of claim 1, wherein CD34+ cells make up at least 48.6 percent of said cultured HPC population.

14. The method of claim 1, wherein CD33+CD34+ cells make up at least 33 percent of said cultured HPC population.

15. The method of claim 1, wherein CD13+ cells make up at least 79 percent of said cultured HPC population.

16. The method of claim 1, wherein CD4low cells make up at least 80 percent of said cultured HPC population.

17. The method of claim 1, wherein said cultured HPC population is substantially free of CD8+, CD20+, and/or CD56+ cells.

18. The method of claim 1, wherein said cultured HPC population is characterized by a ratio of at least 7 to 1 of cells displaying said characteristic associated with a myeloid phenotype to cells not displaying said characteristic.

19. The method of claim 1, wherein said tolerance-inducing activity is induction of tolerance in cytotoxic T-lymphocytes specifically to antigens of said cultured HPC population, wherein said CTLs and said cultured HPC population are allogeneic or xenogeneic with each other.

20. The method of claim 1, wherein said tolerance inducing activity is enhanced per total population in said cultured HPC population relative to said HPC population derived from the donor.

21. The method of claim 1, wherein CD34-negative cells make up at least 50.5 percent of said cultured HPC population.

22. The method of claim 1, wherein said tolerance-inducing activity is a veto activity.

23. A method of transplanting a transplant derived from a donor to a recipient, the method comprising:
(a) administering to the recipient a dose of a cultured HPC population having a tolerance-inducing activity, wherein cells displaying a characteristic associated with a myeloid phenotype make up at least 83.5 percent of said cultured HPC population, and wherein said cultured HPC population is obtained by culturing HPCs derived from the donor; and
(b) transplanting the transplant to the recipient, wherein the donor is allogeneic or xenogeneic with the recipient.

24. The method of claim 23, further comprising the step of conditioning the recipient under sublethal, lethal or supralethal conditions prior to step (b).

25. The method of claim 23, wherein step (a) is performed prior to, concomitantly with or following step (b).

26. The method of claim 23, wherein the transplant is selected from the group consisting of cells, a tissue and an organ.

27. The method of claim 23, wherein said culturing is performed in vitro.

28. The method of claim 23, wherein said cultured HPC population is predominantly CD33+.

29. The method of claim 23, wherein said tolerance inducing activity is enhanced per cell in said cultured HPC population relative to said HPC population derived from the donor.

30. The method of claim 23, wherein the transplant is MHC-mismatched with the recipient, and whereas said dose of said cultured HPC population possesses sufficient tolerance-inducing activity so as to enable engraftment of the transplant.

31. The method of claim 23, wherein said culturing is effected in absence of exogenously added IL-2, TNF-α and/or IFN-γ.

32. The method of claim 23, wherein the recipient and/or the donor is a human.

33. The method of claim 23, wherein said HPC population derived from the donor is a population of purified CD34+ cells.

34. The method of claim 23, wherein said cultured HPC population is characterized by a ratio of at least 7 to 1 of cells displaying said characteristic associated with a myeloid phenotype to cells not displaying said characteristic.

35. The method of claim 23, wherein CD34+ cells make up at least 48.6 percent of said cultured HPC population.

36. The method of claim 23, wherein CD33+CD34+ cells make up at least 33 percent of said cultured HPC population.

37. The method of claim 23, wherein CD13+ cells make up at least 79 percent of said cultured HPC population.

38. The method of claim 23, wherein CD4low cells make up at least 80 percent of said cultured HPC population.

39. The method of claim 23, wherein said cultured HPC population is substantially free of CD8+, CD20+, and/or CD56+ cells.

40. The method of claim 23, wherein said culturing is effected under growth conditions selected so as to induce differentiation into CD33+ cells in said cultured HPC population.

41. The method of claim 23, wherein said characteristic associated with a myeloid phenotype is surface expression of CD33.

42. The method of claim 23, wherein said tolerance-inducing activity is induction of tolerance in cytotoxic T-lymphocytes specifically to antigens of said cultured HPC population, wherein said CTLs and said cultured HPC population are allogeneic or xenogeneic with each other.

43. The method of claim 23, wherein said tolerance inducing activity is enhanced per total population in said cultured HPC population relative to said HPC population derived from the donor.

44. The method of claim 23, wherein CD34-negative cells make up at least 50.5 percent of said cultured HPC population.

45. The method of claim 23, wherein said tolerance-inducing activity is a veto activity.

46. A method of predicting the veto activity of a population of cultured HPCs, the method comprising:
(a) identifying cells displaying a characteristic associated with a myeloid phenotype in the population of cultured HPCs; and
(b) determining within the population of cultured HPCs a ratio between cells displaying said characteristic associated with a myeloid phenotype and cells not displaying said characteristic associated with a myeloid phenotype.

47. The method of claim 46, wherein said step of identifying cells displaying a characteristic associated with a myeloid phenotype in the population of cultured HPCs is effected by detecting cells expressing a myeloid-specific molecule selected from the group consisting of an intracellular protein, a membrane-bound protein, a secreted protein, a messenger RNA (mRNA) transcript, a lipid, a carbohydrate, a hormone and a metabolite.

48. The method of claim 46, wherein said characteristic associated with a myeloid phenotype in the population of cultured HPCs is expression of CD33.

49. The method of claim 46, wherein said step of identifying cells displaying a characteristic associated with a myeloid phenotype in said population of cultured HPCs is effected by a method selected from the group consisting of antibody recognition, ligand recognition and polymerase chain reaction (PCR) amplification.

50. The method of claim 46, wherein said step of identifying cells displaying a characteristic associated with a myeloid phenotype in said population of cultured HPCs is effected by detection of a physical criterion, said physical criterion selected from the group consisting of cellular morphology, cell size, cell density, cellular organelle morphology, cellular organelle size and cytoplasmic light scattering.

51. The method of claim 46, wherein said step of identifying cells displaying a characteristic associated with a myeloid phenotype in the population of cultured HPCs is effected by histological staining or by a functional cellular or biochemical assay.

52. The method of claim 46, further comprising the step of correlating the veto activity of the population of cultured HPCs with said ratio.
